(19) United States
(12) Patent Application Publication
(10) Pub. No.: US 2025/0289038 A1
(43) Pub. Date: Sep. 18, 2025

(54) WASHING AND DISINFECTION APPARATUS AND LID PORTION

(71) Applicant: FUJIFILM Corporation, Tokyo (JP)

(72) Inventors: Yuki HARASAWA, Kanagawa (JP); Keiichiro TAKAHASHI, Kanagawa (JP)

(73) Assignee: FUJIFILM Corporation, Tokyo (JP)

(21) Appl. No.: 19/074,432

(22) Filed: Mar. 9, 2025

(30) Foreign Application Priority Data
Mar. 12, 2024 (JP) 2024-038232

(51) Int. Cl. B08B 3/08; A61L 2/18; B08B 1/14; B08B 3/02

(52) U.S. Cl. CPC B08B 3/08; A61L 2/18; B08B 1/143; A61L 2202/121; A61L 2202/122; A61L 2202/16; A61L 2202/17; A61L 2202/24; B08B 3/02

(57) ABSTRACT

Provided are a washing and disinfection apparatus and a lid portion capable of improving water draining and workability of wiping work on a back surface portion of the lid portion. A washing tank that accommodates a medical instrument, a lid portion (top cover) that covers an opening portion of the washing tank (washing tank opening portion), a recessed portion that is provided in a lid main body portion (cover main body portion) of the lid portion and includes a bottom portion (recessed bottom portion) and a side wall portion that connects the bottom portion and the lid main body portion, and a bottom opening that is formed in the bottom portion and communicates with the inside of the washing tank are provided, in which the side wall portion is an inclined portion having a tapered shape from the lid main body portion toward the bottom portion.

### CROSS-REFERENCE TO RELATED APPLICATION

The present application claims priority under 35 U.S.C § 119 (a) to Japanese Patent Application No. 2024-038232 filed on Mar. 12, 2024, which is hereby expressly incorporated by reference, in its entirety, into the present application.

### BACKGROUND OF THE INVENTION

### 1. Field of the Invention

The present invention relates to a washing and disinfection apparatus for a medical instrument and a lid portion of the washing and disinfection apparatus.

### 2. Description of the Related Art

A washing and disinfection apparatus that washes and disinfects an endoscope, which is a used medical instrument, is known. The washing and disinfection apparatus comprises a washing tank that accommodates a used endoscope. The washing and disinfection apparatus performs washing and disinfection treatment (also referred to as reprocessing treatment) on the used endoscope in the washing tank. In the washing and disinfection apparatus, a top cover (lid portion) that covers an opening portion of the washing tank is provided in order to prevent a washing solution, a disinfectant, and the like from being scattered outside the washing tank during washing and disinfection treatment. The top cover is provided to be rotationally movable between a closed position where the opening portion of the washing tank is covered and an open position where the opening portion of the washing tank is opened with respect to an apparatus main body of the washing and disinfection apparatus.

The top cover is formed with a ventilation path that allows an inside and an outside of the washing tank to communicate with each other and through which a gas flows inside and outside the washing tank. In addition, the top cover is provided with a filter accommodation portion (also referred to as a filter mounting portion) (see JP2022-128907A, JP2015-070947A, and WO2019/014968A). The filter accommodation portion accommodates a deodorizing filter that is disposed in the middle of the ventilation path and deodorizes a gas discharged from the inside of the washing tank to the outside.

The filter accommodation portion described in JP2022-128907A and JP2015-070947A is provided on a back surface portion of the top cover in a convex shape. The filter accommodation portion comprises a vertical wall portion that extends in a vertical direction from a back surface portion of the top cover, a bottom portion that is installed in conjunction with the vertical wall portion and covers a lower surface of the deodorizing filter, and a slit hole for ventilation that is formed in the bottom portion. In addition, a cross section of the slit hole of the filter accommodation portion described in JP2015-070947A is bent in a crank shape.

The filter accommodation portion described in WO2019/014968A is provided on a front surface portion of the top cover on the side opposite to the back surface portion. Here, a protruding portion that protrudes in a convex shape from a back surface portion side to a front surface portion side is formed on a part of the top cover described in WO2019/014968A, and the filter accommodation portion described in WO2019/014968A is provided on the front surface portion side of the protruding portion. A ventilation port is provided in the filter accommodation portion.

### SUMMARY OF THE INVENTION

In a case where the top cover is rotationally moved from the closed position to the open position after the used endoscope is subjected to the washing and disinfection treatment, liquid droplets of the washing solution, the disinfectant, and the like adhered to the back surface portion of the top cover flow down from a front end side to a rear end side of the top cover and then fall into the washing tank from the rear end of the top cover (see JP2022-128907A). In this case, the filter accommodation portion described in JP2022-128907A and JP2015-070947A is provided on the back surface portion of the top cover in a convex shape, and further comprises the vertical wall portion that extends in the vertical direction from the back surface portion. Therefore, the movement of the liquid droplet that flows down toward the rear end side of the top cover is hindered by the vertical wall portion, and the liquid droplet may remain on the back surface portion of the top cover. As a result, the water (liquid) draining of the liquid droplet on the back surface portion of the top cover is deteriorated.

In addition, in a case where wiping (cleaning) work of liquid droplets remaining on the back surface portion of the top cover using a wiping cloth or the like is performed after the used endoscope is subjected to the washing and disinfection treatment, there is a concern that the movement of the cloth or the like may be hindered by the vertical wall portion of the filter accommodation portion and the workability of the wiping work may be deteriorated.

In the washing and disinfection apparatus described in WO2019/014968A, the protruding portion is formed on the front surface portion side of the top cover, but the protruding portion is recessed in a case of being viewed from the back surface portion side of the top cover. Therefore, even in the washing and disinfection apparatus described in WO2019/014968A, in a case where the top cover is rotationally moved from the closed position to the open position after the used endoscope is subjected to the washing and disinfection treatment, there is a concern that liquid droplets that flow down from the front end side to the rear end side of the top cover may be accumulated in the protruding portion. Therefore, the water (liquid) draining of the liquid droplet on the back surface portion of the top cover is deteriorated.

In addition, the protruding portion described in WO2019/014968A is recessed in a direction away from a front side of the washing and disinfection apparatus in a case of being viewed from the front side of the washing and disinfection apparatus in a state where the top cover is rotationally moved to the open position. As a result, in a case where the operator performs the wiping work on the back surface portion of the top cover from the front side of the washing and disinfection apparatus, since it is necessary to extend the hand to the inside of the protruding portion (a recessed portion in a case of being viewed from the operator), the workability of the wiping work is deteriorated.

Aspects of the present invention have been made in view of such circumstances, and an object of the aspects of the present invention is to provide a washing and disinfection apparatus and a lid portion capable of improving water draining and workability of wiping work on a back surface portion of the lid portion.

A washing and disinfection apparatus according to a first aspect of the present invention is a washing and disinfection apparatus for a medical instrument, comprising: a washing tank that accommodates the medical instrument; a lid portion that covers an opening portion of the washing tank; a recessed portion that is provided in a lid main body portion of the lid portion and includes a bottom portion and a side wall portion that connects the bottom portion and the lid main body portion; and a bottom opening that is formed in the bottom portion and communicates with the inside of the washing tank, in which the side wall portion is an inclined portion having a tapered shape from the lid main body portion toward the bottom portion.

With the washing and disinfection apparatus, the water (liquid) draining and the workability of the wiping work of liquid droplets on the back surface portion of the lid portion are improved.

According to a second aspect of the present invention, in the washing and disinfection apparatus according to the first aspect, a tubular portion extending in a direction from the bottom opening toward the washing tank is provided at the bottom portion. As a result, the washing solution or the like in the washing tank is less likely to enter the recessed portion.

According to a third aspect of the present invention, in the washing and disinfection apparatus according to the first aspect or the second aspect, a cover that covers the recessed portion is provided on the lid portion.

According to a fourth aspect of the present invention, in the washing and disinfection apparatus according to the third aspect, the cover is provided with a filter accommodation portion that accommodates a filter.

According to a fifth aspect of the present invention, in the washing and disinfection apparatus according to the fourth aspect, an inner lid that covers at least the bottom opening of the bottom portion is provided between the filter accommodation portion and the bottom portion, and a ventilation space that communicates the filter accommodation portion with the bottom portion is formed in the inner lid. As a result, the gas in the washing tank can be discharged to the outside of the washing tank through the filter.

According to a sixth aspect of the present invention, in the washing and disinfection apparatus according to the fifth aspect, the inner lid has a top surface and an outer side surface, and a first ventilation hole is formed on the outer side surface. Accordingly, the filter accommodation portion and the bottom portion of the recessed portion can communicate with each other.

According to a seventh aspect of the present invention, in the washing and disinfection apparatus according to the fifth aspect or the sixth aspect, an inner lid mounting portion on which the inner lid is mounted is provided in the filter accommodation portion.

According to an eighth aspect of the present invention, in the washing and disinfection apparatus according to any one of the fifth to seventh aspects, the filter accommodation portion accommodates the filter on a surface side opposite to a surface side facing the bottom portion of the inner lid.

According to a ninth aspect of the present invention, in the washing and disinfection apparatus according to any one of the fourth to eighth aspects, the cover is provided with an outer lid that covers the filter accommodation portion. As a result, the filter is prevented from falling off from the filter accommodation portion.

According to a tenth aspect of the present invention, in the washing and disinfection apparatus according to the ninth aspect, a second ventilation hole is formed in the outer lid. Accordingly, the inside and the outside of the filter accommodation portion communicate with each other.

According to an eleventh aspect of the present invention, the washing and disinfection apparatus according to any one of the first to tenth aspects further comprises an apparatus main body that performs washing and disinfection of the medical instrument accommodated in the washing tank, in which the lid portion is attached to the apparatus main body to be rotationally movable between an open position where the opening portion is opened and a closed position where the opening portion is closed.

A lid portion according to a twelfth aspect of the present invention is a lid portion that covers an opening portion of a washing tank in which a medical instrument is accommodated in a washing and disinfection apparatus for a medical instrument, the lid portion comprising: a lid main body portion; a recessed portion that is provided in the lid main body portion and includes a bottom portion and a side wall portion that connects the bottom portion and the lid main body portion; and a bottom opening that is formed in the bottom portion, in which the side wall portion is an inclined portion having a tapered shape from the lid main body portion toward the bottom portion.

According to the aspects of the present invention, the water draining and the workability of the wiping work on the back surface portion of the lid portion are improved.

### BRIEF DESCRIPTION OF THE DRAWINGS

FIG. 1 is an external perspective view of a washing and disinfection apparatus in a state where a top cover is rotationally moved to a closed position.

FIG. 2 is an external perspective view of the washing and disinfection apparatus in a state where the top cover is rotationally moved to an open position.

FIG. 3 is an external perspective view of the top cover and a rear cover.

FIG. 4 is an exploded perspective view showing a state where a packing, an inner lid, the rear cover, and a bracket are detached from the top cover.

FIG. 5 is an external perspective view of the top cover as viewed from a front surface portion side.

FIG. 6 is an external perspective view of the top cover as viewed from a back surface portion side.

FIG. 7 is an external perspective view showing a state where the packing is detached from the top cover shown in FIG. 6.

FIG. 8 is an enlarged perspective view of a recessed portion as viewed from the front surface portion side of the top cover.

FIG. 9 is an enlarged perspective view of the recessed portion as viewed from the back surface portion side of the top cover.

FIG. 10 is a cross-sectional view of the recessed portion taken along the line X-X in FIG. 8.

FIG. 11 is a cross-sectional view of the recessed portion taken along the line XI-XI in FIG. 8.

FIG. 12 is an enlarged cross-sectional view of the top cover of a comparative example in a state of being rotationally moved to the open position.

FIG. 13 is a perspective view showing a state where the bracket is attached to a cover attachment portion of the top cover.

FIG. 14 is an external perspective view of the packing attached to the recessed portion.

FIG. 15 is an external perspective view of an inner lid attached to the recessed portion.

FIG. 16 is an external perspective view of the inner lid as viewed from a front surface portion side.

FIG. 17 is an external perspective view of the inner lid as viewed from a back surface portion side.

FIG. 18 is a cross-sectional view taken along the line XVIII-XVIII in FIG. 15.

FIG. 19 is an external perspective view of the rear cover and an outer lid.

FIG. 20 is an exploded perspective view showing a state where a deodorizing filter and the outer lid are detached from the rear cover.

FIG. 21 is an external perspective view of the rear cover as viewed from a front surface portion side.

FIG. 22 is an external perspective view of the rear cover as viewed from a back surface portion side.

FIG. 23 is a top view of the rear cover.

FIG. 24 is a top view of the rear cover before the deodorizing filter is accommodated.

FIG. 25 is a top view of the rear cover after the deodorizing filter is accommodated.

FIG. 26 is a top view of the rear cover in a state where the outer lid is rotationally moved to an open position.

FIG. 27 is a top view of the rear cover in a state where the outer lid is rotationally moved to a closed position.

### DESCRIPTION OF THE PREFERRED EMBODIMENTS

### Washing and Disinfection Apparatus 10

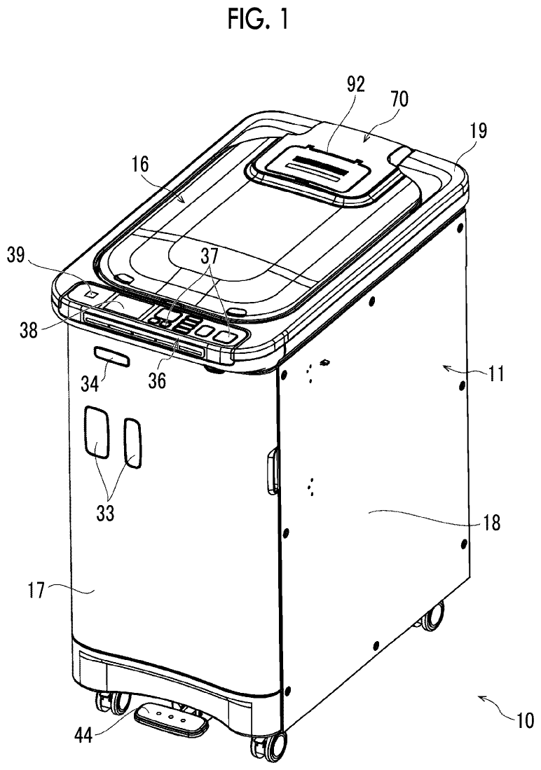

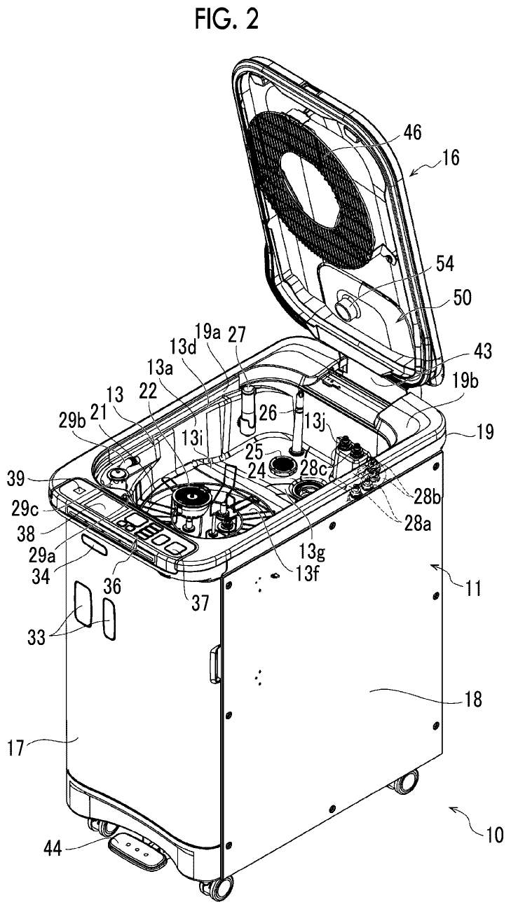

FIG. 1 is an external perspective view of a washing and disinfection apparatus 10 in a state where a top cover 16 is rotationally moved to a closed position. FIG. 2 is an external perspective view of the washing and disinfection apparatus 10 in a state where the top cover 16 is rotationally moved to an open position.

As shown in FIGS. 1 and 2, the washing and disinfection apparatus 10 performs washing and disinfection treatment on an endoscope (not shown, the same applies hereinafter), which is a used medical instrument.

The washing and disinfection apparatus 10 comprises a box-shaped apparatus main body 11. A washing tank 13 for accommodating a used endoscope is provided in an upper portion of the apparatus main body 11. A washing solution and a disinfectant are supplied to the washing tank 13 from the apparatus main body 11. The washing tank 13 is a water tank having an open upper portion, and is formed of, for example, a metal material having excellent heat resistance and corrosion resistance, such as stainless steel. The top cover 16 that covers a washing tank opening portion 13a, which is an opening portion of the washing tank 13, is attached to the apparatus main body 11.

The apparatus main body 11 has a chassis (not shown, the same applies hereinafter). In addition to the washing tank 13 and the top cover 16, although not shown, a pipe for supplying a detergent and a disinfectant to the washing tank 13, a pump, a solenoid valve, a tank for storing a disinfectant, and the like are attached to the chassis. The outer periphery of the chassis is covered with exterior members such as a front panel 17, a side panel 18, and an upper panel 19. The front panel 17 constitutes a front surface portion of the washing and disinfection apparatus 10, the side panel 18 constitutes a side surface portion of the washing and disinfection apparatus 10, and the upper panel 19 constitutes an upper surface portion of the washing and disinfection apparatus 10.

The front panel 17 is attached to the chassis through a hinge (not shown) at a side end portion thereof, and is openable and closable. A storage tray (not shown) is provided inside the front panel 17. A detergent tank and an alcohol tank (not shown) are stored in the storage tray. A detergent used for washing the endoscope is stored in the detergent tank. The alcohol tank stores alcohol that is flowed into various known channels of the endoscope after the endoscope is subjected to the washing and disinfection treatment. In addition, a transparent window 33 for viewing the remaining amount of the liquid in each tank is attached to the front panel 17.

A supply bottle (not shown) in which a concentrated solution obtained by concentrating a known disinfectant (see JP2022-128907A) is stored is replaceably stored in the storage tray (not shown). The supply bottle is connected to a disinfectant tank (not shown) provided in the chassis, and supplies the concentrated solution into the disinfectant tank. The concentrated solution is used after being diluted with water in a disinfectant tank to an appropriate concentration.

A paper discharge port 34 is provided above the transparent window 33 of the front panel 17. A printer (not shown) is disposed at the back of the paper discharge port 34. The paper discharge port 34 discharges a print on which washing history information is printed by the printer. The washing history information is, for example, information such as the date and time of performing the washing and disinfection treatment, the name of an operator, and an identification (ID) of the endoscope that has been subjected to the washing and disinfection treatment. The print on which the washing history information is printed is used for checking and managing the washing and disinfection result of the endoscope.

An operation unit 36 is provided at a front end portion of the upper panel 19. The operation unit 36 is provided with a plurality of operation buttons 37 for inputting various operation instructions, a display 38 for performing various displays, and a tag reader 39. The operation buttons 37 are, for example, a start button for instructing the start of the washing and disinfection treatment, a stop button for instructing the emergency stop, an operation key for operating an operation screen displayed on the display 38, and the like.

The display 38 is, for example, a liquid crystal display, and displays the operation screen including a selection screen for selecting a menu for the washing and disinfection treatment and a setting screen for performing various settings. In addition, the display 38 displays various kinds of information including the progress status and the remaining time of the washing and disinfection treatment, a warning message in a case where a trouble occurs.

The tag reader 39 communicates with a radio frequency identification (RFID) tag provided in the endoscope and an RFID tag provided in a nameplate of the operator in a noncontact manner to read information in the RFID tags.

The upper panel 19 has a frame shape in which a panel opening portion 19a that is an opening portion through which the washing tank opening portion 13a is exposed is formed. A peripheral edge portion 13d that is orthogonal to a side surface that rises vertically from the bottom portion of the washing tank 13 and extends in a horizontal direction is formed at the upper end of the washing tank opening portion 13a. The peripheral edge portion 13d and the upper panel 19 are joined to each other such that a gap through which a gas and a liquid leak from a boundary between the peripheral edge portion 13d and the upper panel 19 does not occur. An inclined surface 19b inclined toward the washing tank opening portion 13a is formed in the panel opening portion 19a so that the liquid flows toward the washing tank 13 by the action of gravity.

A net 21 and a small article washing basket 22 are attached to a bottom front portion 13f which is a front portion (a portion on the front panel 17 side) of the bottom portion of the washing tank 13. The net 21 creates a gap in which the liquid flows between the endoscope and the bottom surface of the washing tank 13 so that the area of contact between the liquid supplied to the washing tank 13 and the outer surface of the endoscope is increased. The small article washing basket 22 accommodates small article components (not shown, the same applies hereinafter) such as an air supply/water supply button and a suction button detached from the endoscope.

A slope 13i that descends from the bottom front portion 13f toward a bottom rear portion 13g, which is a rear portion (a portion on a cover attachment portion 43 side, which will be described later) of the bottom portion of the washing tank 13, is formed at a boundary between the bottom front portion 13f and the bottom rear portion 13g. The bottom rear portion 13g is lower than the bottom front portion 13f by one step. The slope 13i improves the water draining from the bottom front portion 13f to the bottom rear portion 13g.

Although not shown, a vibration plate for ultrasonic washing, a back surface washing nozzle for washing a back surface portion of the top cover 16, a small article washing nozzle for washing small articles in the small article washing basket 22, a temperature sensor for measuring a temperature of a liquid stored in the washing tank 13, and the like are provided in a central portion of the bottom front portion 13f (see JP2015-070947A).

A detergent supply nozzle 29a, a disinfectant supply nozzle 29b, and a float switch 29c are provided at a peripheral edge portion 13d of the substantially front side of the bottom front portion 13f. The detergent supply nozzle 29a supplies a detergent stored in the detergent tank (not shown) to the washing tank 13. Body fluids and excrement adhered to the used endoscope are washed away by a washing solution in which water and a detergent are mixed. The disinfectant supply nozzle 29b is a metal pipe bent toward the inside of the washing tank 13, and supplies a disinfectant stored in the disinfectant tank (not shown) to the inside of the washing tank 13. Pathogenic bacteria and viruses that are not washed away by the washing solution are killed or inactivated by the disinfectant. The float switch 29c is an overflow detection switch for preventing the liquid supplied to the washing tank 13 from overflowing to the peripheral edge portion 13d.

A waste liquid outlet 24 and a circulation port 25 are provided on a bottom surface of the bottom rear portion 13g. The waste liquid outlet 24 discharges the used washing solution, disinfectant, and the water used for rinsing from the washing tank 13. The circulation port 25 is provided to circulate the liquid stored in the washing tank 13 and to generate a water flow in the liquid in the washing tank 13. The liquid suctioned from the circulation port 25 is re-supplied to the washing tank 13 from the back surface washing nozzle and the small article washing nozzle (not shown) described above and the water supply nozzle 26 to be described later.

A water supply nozzle 26 that supplies water to the washing tank 13 is installed to stand behind the circulation port 25 of the bottom rear portion 13g. In addition, a water level sensor 27 is attached to a side surface of the bottom rear portion 13g on the circulation port 25 side. The water level sensor 27 detects a position (water level) of a liquid level of a liquid stored in the washing tank 13.

A terrace portion 13j having a height up to the middle of the washing tank 13 is provided at a corner of the bottom rear portion 13g on the waste liquid outlet 24 side.

On the upper surface of the terrace portion 13j, an airtightness test connector 28a, a main channel washing connector 28b, and a sub-channel washing connector 28c are provided in order from the front. Each of the connectors 28a to 28c is a fluid supply connector for supplying a fluid to the inside of the endoscope. Since the configuration and the function of each of the connectors 28a to 28c are known technologies (see JP2015-070947A), the specific description thereof will be omitted here.

### Top Cover 16

The top cover 16 corresponds to a lid portion according to an embodiment of the present invention. The top cover 16 is formed of, for example, a transparent or translucent plastic material so that the state of the washing tank 13 can be visually recognized from the outside. The top cover 16 is attached to the apparatus main body 11 through a cover attachment portion 43 provided on a rear side (proximal end side) of the top cover 16. The cover attachment portion 43 is rotationally moved about a rotational movement shaft (not shown) provided in the apparatus main body 11. Accordingly, the top cover 16 is rotationally movable between an open position (see FIG. 2) where the washing tank opening portion 13a is opened and a closed position (see FIG. 1) where the washing tank opening portion 13a is closed, with the cover attachment portion 43 (rotational movement shaft) as a fulcrum.

In a state where the top cover 16 is in the closed position, a lower surface portion of the top cover 16 facing the inside of the washing tank 13 is referred to as a “back surface portion”, and an upper surface portion on the side opposite to the back surface portion is referred to as a “front surface portion”. In addition, the “front portion of ˜” and the “front side of ˜” indicate a “portion of ˜ on the front panel 17 side” and a “direction toward ˜ on the front panel 17 side” in a state where the top cover 16 is in the closed position. Further, the “rear portion of ˜” and the “rear side of ˜” indicate a “portion of ˜ on the cover attachment portion 43 side” and a “direction toward ˜ on the cover attachment portion 43 side” in a state where the top cover 16 is in the closed position.

The top cover 16 is opened and closed by an electric actuator (motor or the like) (not shown). The operation instruction for opening and closing the top cover 16 is input by a stepping operation on the foot switch 44 disposed below the front panel 17. In a case where the foot switch 44 is stepped on in a state where the top cover 16 is in the closed position, the top cover 16 is rotationally moved to the open position by the electric actuator. Conversely, in a case where the foot switch 44 is stepped on in a state where the top cover 16 is in the open position, the top cover 16 is rotationally moved to the closed position by the electric actuator.

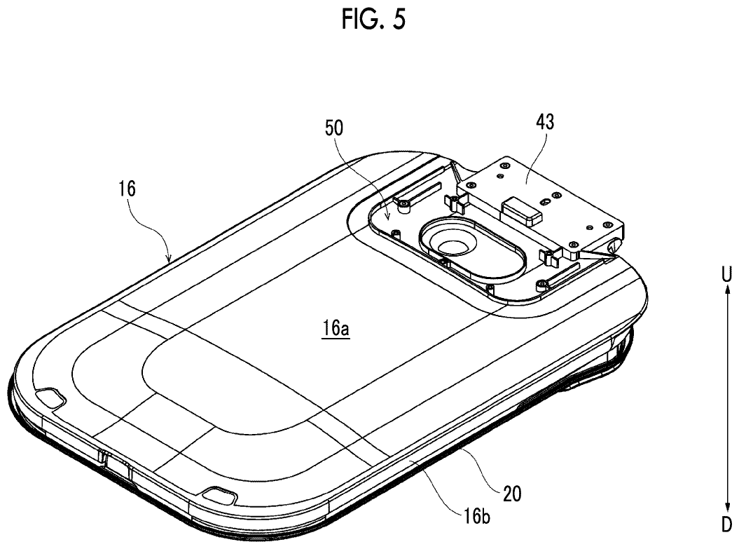

A rear cover 70, which will be described later, is provided on a rear portion of the front surface portion of the top cover 16. Further, an outer lid 92, which will be described later, is provided on the rear cover 70. A net 46 is provided on a front portion of the back surface portion of the top cover 16. The net 46 is disposed at a position facing the net 21 disposed on the bottom front portion 13f in a state where the top cover 16 is in the closed position. The net 46 presses the endoscope accommodated in the washing tank 13 from above and sinks the endoscope below the liquid level. Although it will be described in detail later, a bottom portion of the recessed portion 50 (see FIG. 5) and a cylindrical portion 54 provided at the bottom portion of the recessed portion 50 are provided in a rear portion of the back surface portion of the top cover 16.

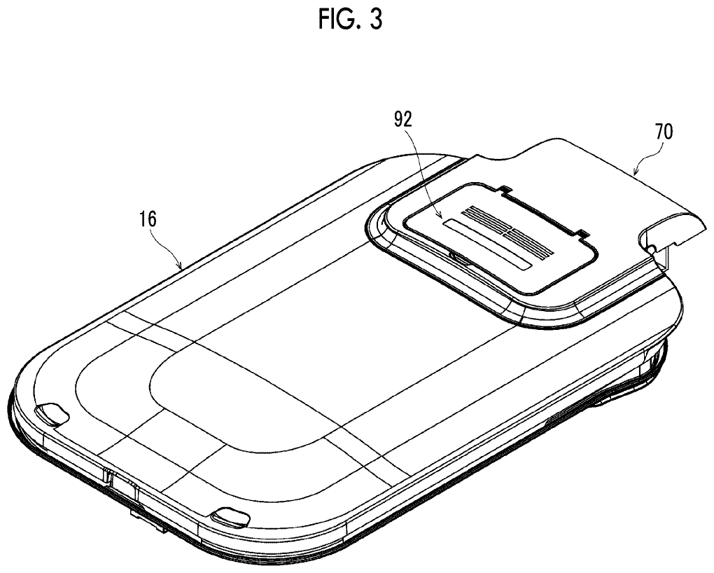

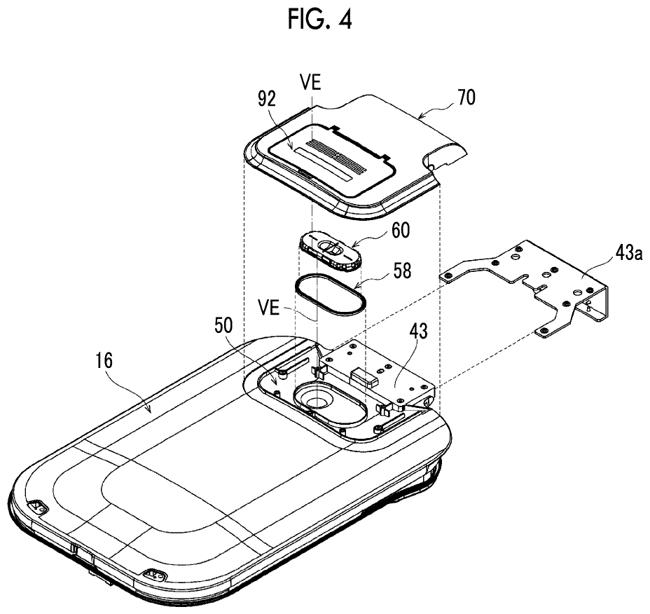

FIG. 3 is an external perspective view of the top cover 16 and the rear cover 70. FIG. 4 is an exploded perspective view showing a state where a packing 58, an inner lid 60, the rear cover 70, and a bracket 43a are detached from the top cover 16.

As shown in FIGS. 3 and 4, a recessed portion 50 is provided in the rear portion of the front surface portion of the top cover 16. Further, the cover attachment portion 43 is provided on a rear side of the recessed portion 50. The rear cover 70 is provided on the recessed portion 50.

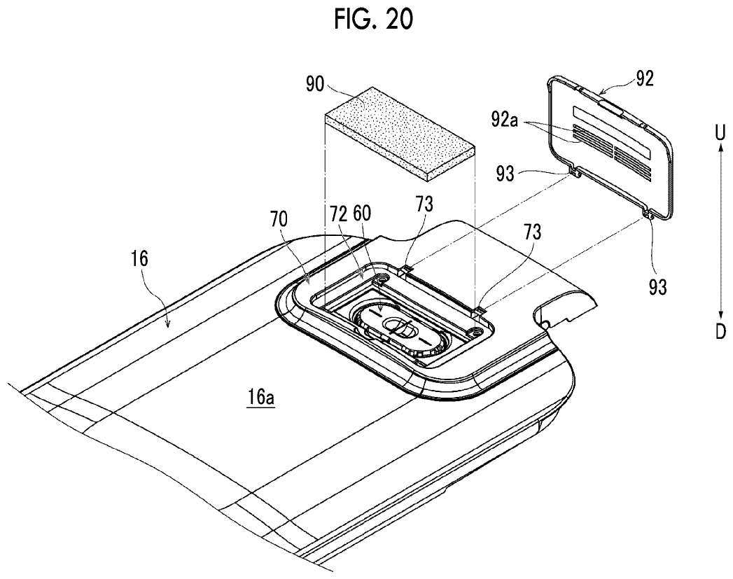

The outer lid 92 is attached to the rear cover 70 to be rotationally movable, and the deodorizing filter 90 (see FIG. 20), which will be described later, is accommodated inside the outer lid 92 in the rear cover 70.

The packing 58, the inner lid 60, and the bracket 43a are provided between the top cover 16 and the rear cover 70. A ventilation path VE that allows the inside and the outside of the washing tank 13 to communicate with each other is formed in the recessed portion 50 of the top cover 16, the inner lid 60, the rear cover 70, and the outer lid 92. A gas in the washing tank 13 is discharged to the outside through the ventilation path VE.

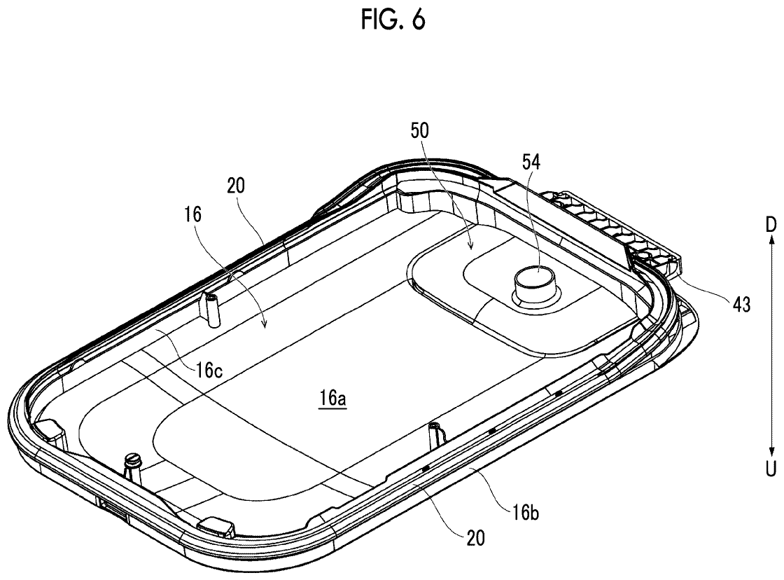

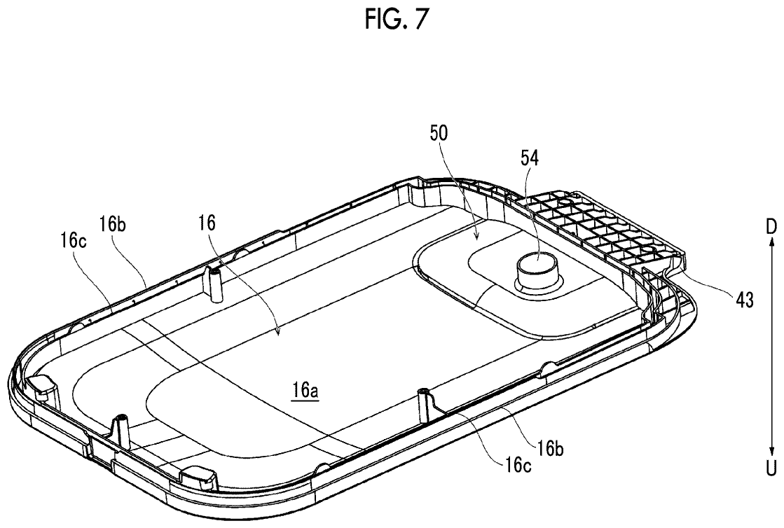

FIG. 5 is an external perspective view of the top cover 16 as viewed from a front surface portion side. FIG. 6 is an external perspective view of the top cover 16 as viewed from a back surface portion side. FIG. 7 is an external perspective view showing a state where a packing 20 is detached from the top cover 16 shown in FIG. 6. The U direction in the drawing is a direction from the back surface portion side to the front surface portion side of the top cover 16 (upward direction in a case where the top cover 16 is in the closed position). In addition, a D direction in the drawing is a direction from the front surface portion side to the back surface portion side of the top cover 16 [a downward direction (direction toward the washing tank 13 side) in a case where the top cover 16 is in the closed position].

As shown in FIGS. 5 to 7, the top cover 16 comprises a cover main body portion 16a having a substantially dome shape bulging in the U direction, and a cover outer peripheral portion 16b that is formed along an outer periphery of the cover main body portion 16a and extends in the D direction from the outer periphery. The cover main body portion 16a corresponds to a lid main body portion according to the embodiment of the present invention and faces the washing tank 13 in a case where the top cover 16 is in the closed position. By forming the cover main body portion 16a in a substantially dome shape, the liquid jetted upward from the above-described bottom front portion 13f easily spreads over the entire washing tank 13.

A rib 16c (see FIG. 7) is formed on a back surface portion of the cover main body portion 16a. The rib 16c is formed along the inside of the cover outer peripheral portion 16b and has a shape extending in the D direction from the back surface portion of the cover main body portion 16a. The packing 20 (see FIG. 6) is attached to the top cover 16 so as to cover the end portions of the cover outer peripheral portion 16b and the rib 16c on the D direction side.

The rib 16c reinforces the cover outer peripheral portion 16b of the top cover 16 formed of a plastic material. The packing 20 is formed of an elastic material such as rubber and seals the washing tank opening portion 13a in a state where the top cover 16 is rotationally moved to the closed position. Therefore, the gas in the washing tank 13 is discharged to the outside through the ventilation path VE in a state where the top cover 16 is rotationally moved to the closed position.

### Recessed Portion 50

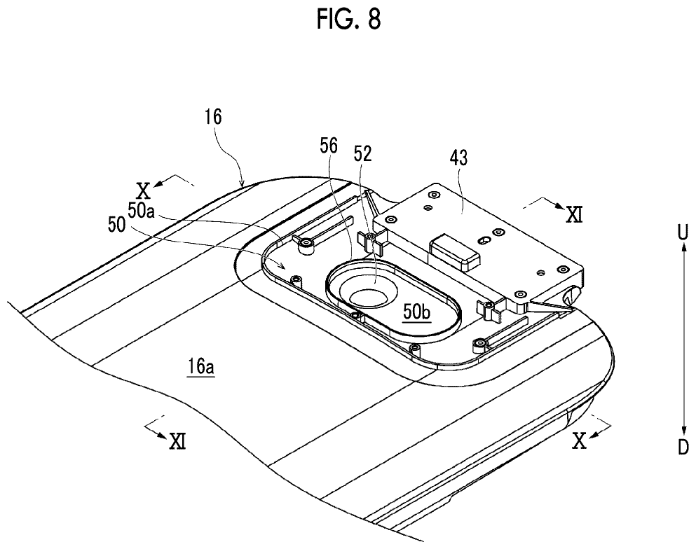

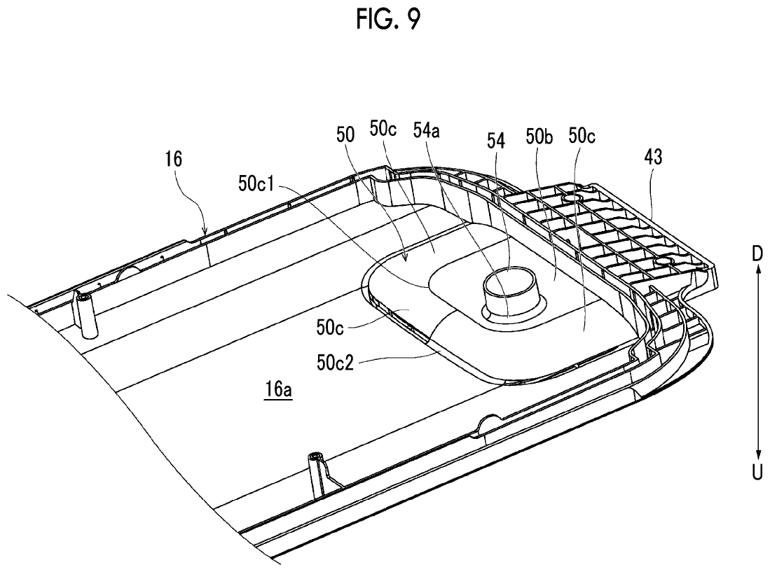

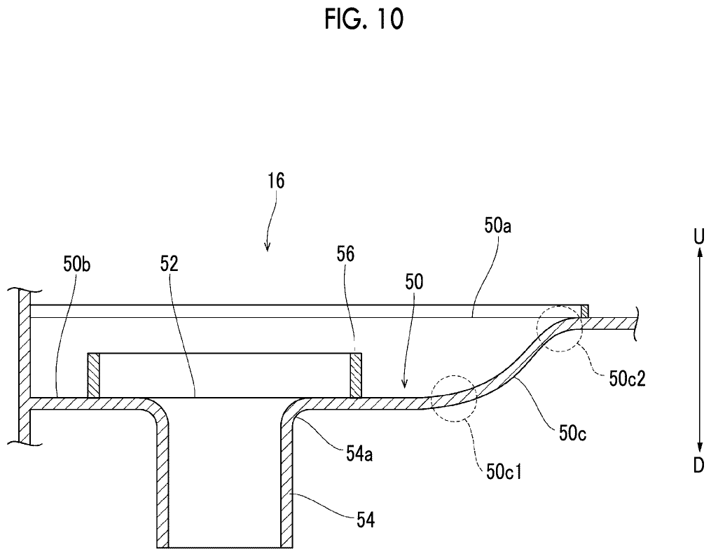

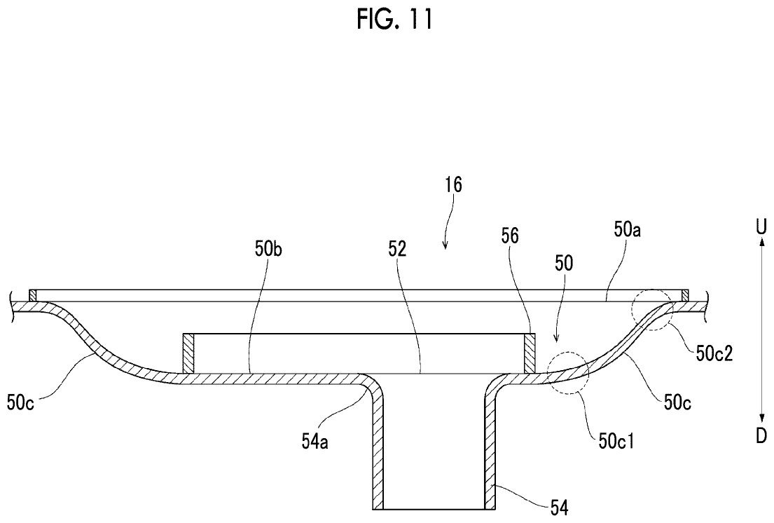

FIG. 8 is an enlarged perspective view of the recessed portion 50 as viewed from the front surface portion side of the top cover 16. FIG. 9 is an enlarged perspective view of the recessed portion 50 as viewed from the back surface portion side of the top cover 16. FIG. 10 is a cross-sectional view of the recessed portion 50 taken along the line X-X in FIG. 8. FIG. 11 is a cross-sectional view of the recessed portion 50 taken along the line XI-XI in FIG. 8.

As shown in FIGS. 8 to 11, the recessed portion 50 is formed by recessing a part of the rear portion of the top cover 16 in the D direction to form, for example, a ship bottom shape or a bathtub shape, and is formed integrally with the top cover 16. For example, in the present embodiment, the recessed portion 50 is formed such that a recessed opening portion 50a, which is an opening portion of the recessed portion 50, is opened in the front surface portion of the top cover 16, and a recessed bottom portion 50b, which is a bottom portion of the recessed portion 50, is located on the D direction side, that is, on the washing tank 13 side with respect to the recessed opening portion 50a. Therefore, the recessed bottom portion 50b is provided at a position closer to the D direction side than the cover main body portion 16a in a case of being viewed from the back surface portion side of the top cover 16. Although not shown, the recessed portion 50 may be subjected to grain processing. In addition, the recessed portion 50 may be provided separately from the cover main body portion 16a.

A bottom opening 52 communicating with the inside of the washing tank 13 is formed in the recessed bottom portion 50b. The cylindrical portion 54 (corresponding to a tubular portion according to the embodiment of the present invention) extending to the D direction side, that is, the direction toward the washing tank 13 is formed from the bottom opening 52. The connection portion 54a between the cylindrical portion 54 and the recessed bottom portion 50b is formed in a curved surface shape. The cylindrical portion 54 is extended to the D direction side (washing tank 13 side) from the bottom opening 52, so that the washing solution or the like in the washing tank 13 is less likely to enter the recessed portion 50. In addition, by forming the connection portion 54a in a curved surface shape, it is easy to perform wiping work for the washing solution or the like adhered to the connection portion 54a. Further, since the cylindrical portion 54 has a cylindrical shape, the cylindrical portion 54 is favorable in the water (liquid) draining.

The number of the bottom openings 52 and the number of the cylindrical portions 54 may be plural. In addition, a tubular portion, instead of the cylindrical portion 54, having various shapes may extend to the D direction side from the bottom opening 52. Further, the shape of the connection portion 54a may also be appropriately changed.

A first wall portion 56 that protrudes to the U direction side (rear cover 70 side) to include the bottom opening 52 is formed on the front surface portion side of the recessed bottom portion 50b. The first wall portion 56 is formed in an oval shape (here, a shape including a linear portion such as a track of an athletic field or an elliptical shape) in a case of being viewed from the front surface portion side of the top cover 16. The shape of the first wall portion 56 is not limited to the oval shape, and various shapes such as an elliptical shape can be adopted. In addition, in the present embodiment, the bottom opening 52 is formed at a position deviated from the center position of the first wall portion 56, but the formation position of the bottom opening 52 is not particularly limited. A region surrounded by the first wall portion 56 on the recessed bottom portion 50b is a mounting portion on which the packing 58 and the inner lid 60, which will be described later, are mounted.

The area of the recessed bottom portion 50b is smaller than the area of the recessed opening portion 50a. Therefore, a side wall portion of the recessed portion 50 connecting the recessed bottom portion 50b and the cover main body portion 16a is an inclined portion 50c having a tapered shape from the cover main body portion 16a toward the recessed bottom portion 50b in a case of being viewed from the back surface portion side of the top cover 16. Accordingly, the inclined portion 50c is inclined to approach the outer periphery of the recessed bottom portion 50b as the inclined portion 50c moves toward the D direction side. In addition, the inclined portion 50c is formed in a curved surface shape bulging to the substantially D direction side (a convex curved surface shape in a case of being viewed from the back surface portion side of the top cover 16). Furthermore, the connection portion 50cl between the inclined portion 50c and the recessed bottom portion 50b, and the connection portion 50c2 between the inclined portion 50c and the cover main body portion 16a are also formed in a curved surface shape.

In this way, by providing the inclined portion 50c in the recessed portion 50 of the top cover 16, even in a case where the top cover 16 is rotationally moved to the open position after the endoscope is subjected to the washing and disinfection treatment, the movement of the liquid droplet that flows down from the front portion to the rear portion of the back surface portion of the top cover 16 is less likely to be hindered by the recessed portion 50. As a result, the water (liquid) draining of the liquid droplets on the back surface portion of the top cover 16 is improved. In addition, since the recessed portion 50 is bulged in the D direction in a case of being viewed from the back surface portion side of the top cover 16, the liquid droplet is also prevented from being accumulated in the recessed portion 50. As a result, the water draining on the back surface portion of the top cover 16 is further improved.

In addition, by providing the inclined portion 50c, in a case where the wiping work on the top cover 16 is performed after the endoscope is subjected to the washing and disinfection treatment, the movement of the wiping cloth or the like is less likely to be hindered by the recessed portion 50. As a result, the workability of the wiping work on the back surface portion of the top cover 16 is improved. Furthermore, in a state where the top cover 16 is rotationally moved to the open position, the recessed bottom portion 50b and the inclined portion 50c are located on the front side of the operator with respect to the cover attachment portion 43 in a case of being viewed from an operator who is located on the substantially front side of the washing and disinfection apparatus 10. Therefore, the hand of the operator easily reaches the recessed bottom portion 50b and the inclined portion 50c, and the workability of the wiping work is improved.

Further, a washing solution or the like jetted from the back surface washing nozzle (not shown) of the bottom front portion 13f toward the back surface portion of the top cover 16 during the washing and disinfection treatment on the endoscope is moved along the back surface portion of the top cover 16 having a substantially dome shape so as to spread over the entire back surface portion. In this case, in a case where all the side wall portions of the recessed portion 50 are vertical wall portions as described in JP2022-128907A and JP2015-070947A, the washing solution or the like that has collided with the vertical wall portion is blocked and is likely to fall into the washing tank 13. Therefore, the washing solution or the like is less likely to spread over the entire back surface portion of the top cover 16. On the other hand, in the present embodiment, since the side wall portion of the recessed portion 50 is formed as the inclined portion 50c, the washing solution or the like that moves along the back surface portion of the top cover 16 easily moves around the recessed portion 50. As a result, the washing solution or the like easily spread over the entire back surface portion of the top cover 16.

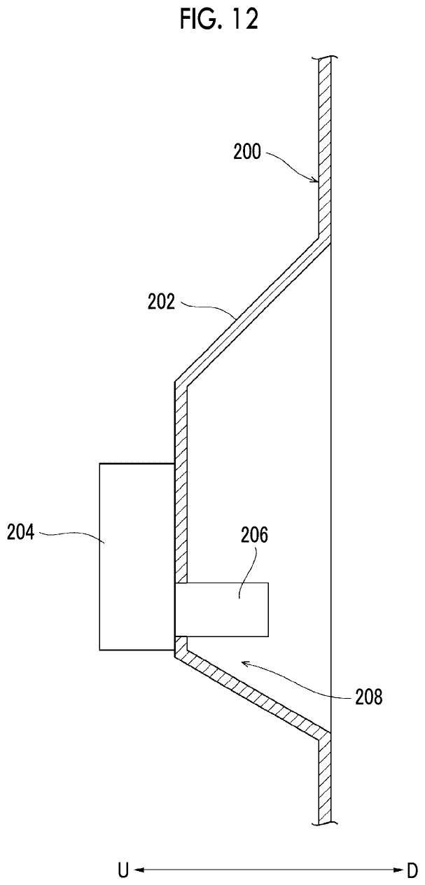

FIG. 12 is an enlarged cross-sectional view of a top cover 200 of a comparative example in a state of being rotationally moved to the open position. As shown in FIG. 12, a protruding portion 202 that is formed in a convex shape from the back surface portion side toward the front surface portion side (U direction side) of the top cover 200 is provided in a part of the top cover 200 of the comparative example, as in the invention described in WO2019/014968A. A filter accommodation portion 204 that accommodates a deodorizing filter 90 (see FIG. 20), which will be described later, is provided on a front surface portion side of the protruding portion 202.

The filter accommodation portion 204 is provided with a ventilation tubular portion 206 that penetrates the top cover 200 and communicates with the inside of the washing tank 13. The ventilation tubular portion 206 further extends from the back surface portion of the top cover 200 to the direction side toward the inside of the washing tank 13 (D direction side). In addition, although not shown, a ventilation path communicating with the outside is also formed in the filter accommodation portion 204. Accordingly, it is possible to discharge the gas in the washing tank 13 to the outside through the ventilation tubular portion 206 and the filter accommodation portion 204.

Since the top cover 200 of the comparative example is formed with the protruding portion 202 that is recessed in a direction opposite to the recessed portion 50 of the present embodiment, in a case where the top cover 200 is rotationally moved to the open position, the liquid droplets that flow down from the front portion to the rear portion of the top cover 200 are accumulated in the protruding portion 202. As a result, the water (liquid) draining on the back surface portion of the top cover 200 is deteriorated. In addition, since the liquid droplet enters a narrow gap 208 between the side wall portion of the protruding portion 202 and the ventilation tubular portion 206, the water draining is deteriorated, and it is difficult for the operator to perform the wiping work of the liquid droplet.

In the present embodiment in contrast to the comparative example, the water (liquid) draining and the workability of the wiping work of the liquid droplets on the back surface portion of the top cover 16 can be improved by providing the recessed portion 50 in a part of the cover main body portion 16a of the top cover 16 and further providing the inclined portion 50c that connects the cover main body portion 16a and the recessed bottom portion 50b. In addition, by forming the inclined portion 50c and the connection portions 50cl and 50c2 in a curved surface shape, the water draining and the workability of the wiping work are further improved.

In the present embodiment, the inclined portions 50c are formed on the front side and the lateral side of the recessed bottom portion 50b in a case being viewed from the back surface portion side of the top cover 16. However, the inclined portions 50c may also be formed on the rear side of the recessed bottom portion 50b. In addition, it is not necessary to form all the side wall portions of the recessed portion 50 in the inclined portion 50c, and at least a part of the side wall portions may be formed in the inclined portion 50c. Further, in the present embodiment, the inclined portion 50c and the connection portions 50cl and 50c2 are formed in a curved surface shape, but these shapes are not particularly limited.

### Bracket 43a

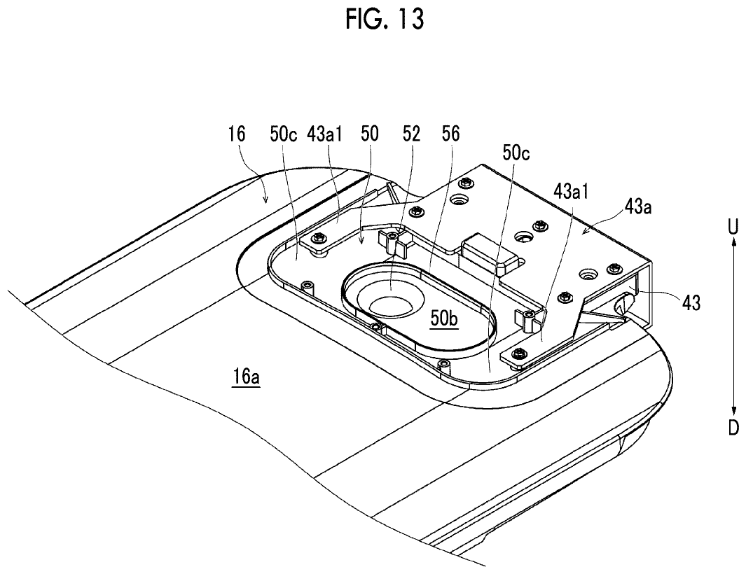

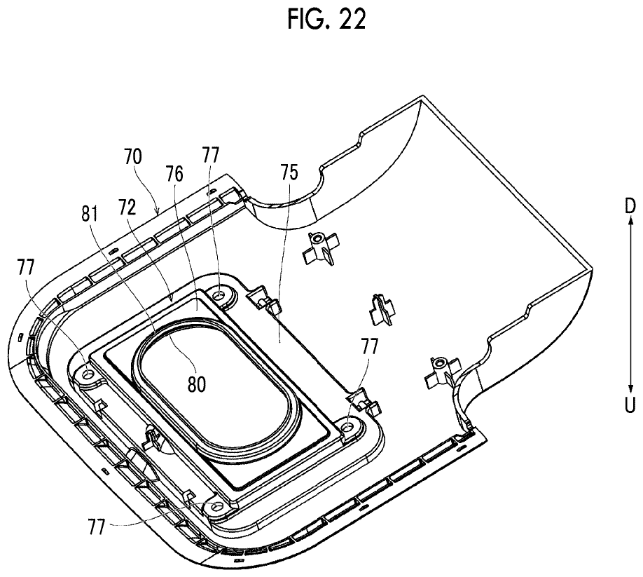

FIG. 13 is a perspective view showing a state where the bracket 43a is attached to the cover attachment portion 43 of the top cover 16. As shown in FIG. 13, the bracket 43a is formed of, for example, a metal material. A rear portion of the bracket 43a is screw-fixed to the cover attachment portion 43. In addition, two bracket distal end portions 43al are formed at a front portion of the bracket 43a. The bracket distal end portion 43al is screw-fixed to the inclined portion 50c in a space formed between the inclined portion 50c and the rear cover 70 [a side region of an outer recessed portion 75 (see FIG. 22)], which will be described later. As a result, the recessed portion 50 can be reinforced with the bracket 43a.

### Packing 58

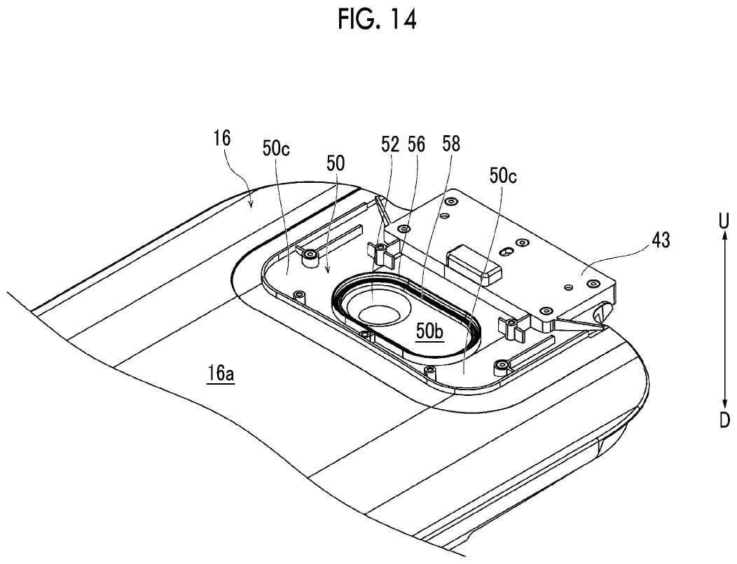

FIG. 14 is an external perspective view of the packing 58 attached to the recessed portion 50. As shown in FIG. 14, the packing 58 is formed in a shape (here, an oval shape) along the inner periphery of the first wall portion 56, and is attached to the inner peripheral side of the first wall portion 56 on the recessed bottom portion 50b. Accordingly, the washing solution or the like in the washing tank 13 is prevented from leaking from the gap between the first wall portion 56 and the inner lid 60, which will be described later.

### Inner Lid 60

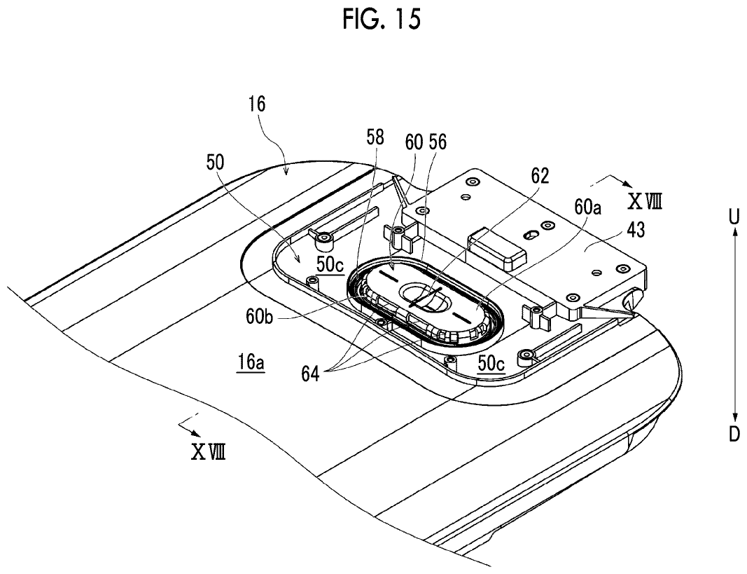

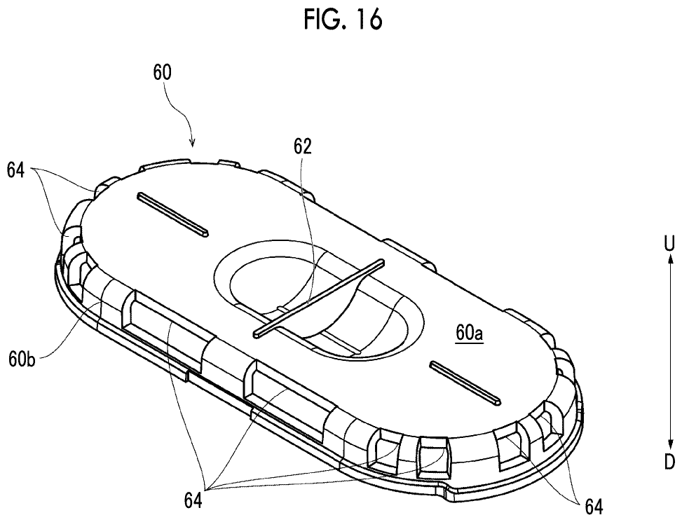

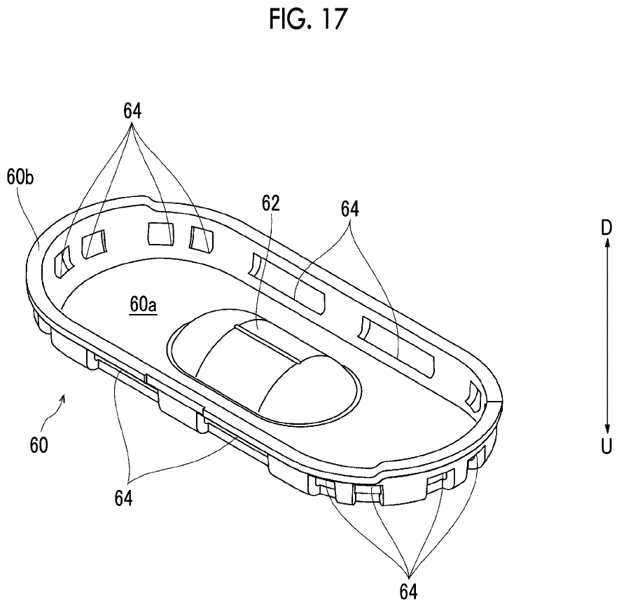

FIG. 15 is an external perspective view of the inner lid 60 attached to the recessed portion 50. FIG. 16 is an external perspective view of the inner lid 60 as viewed from a front surface portion side. FIG. 17 is an external perspective view of the inner lid 60 as viewed from a back surface portion side.

As shown in FIGS. 15 to 17, the inner lid 60 is formed in a shape (here, an oval shape) along the inner periphery of the packing 58 (first wall portion 56) in a case of being viewed from the front surface portion side of the top cover 16. The inner lid 60 is attached on the inner peripheral side of the packing 58 and on the recessed bottom portion 50b to cover at least the bottom opening 52.

The inner lid 60 has a top surface 60a having an oval shape and an outer side surface 60b extending from the outer periphery of the top surface 60a to the D direction side. A gripping portion 62 that is gripped by the operator in a case where the inner lid 60 is detached and attached is formed on the top surface 60a. A plurality of ventilation holes 64 (corresponding to first ventilation holes according to the embodiment of the present invention) are formed in the outer side surface 60b. The shape and the number of the ventilation holes 64 are not particularly limited.

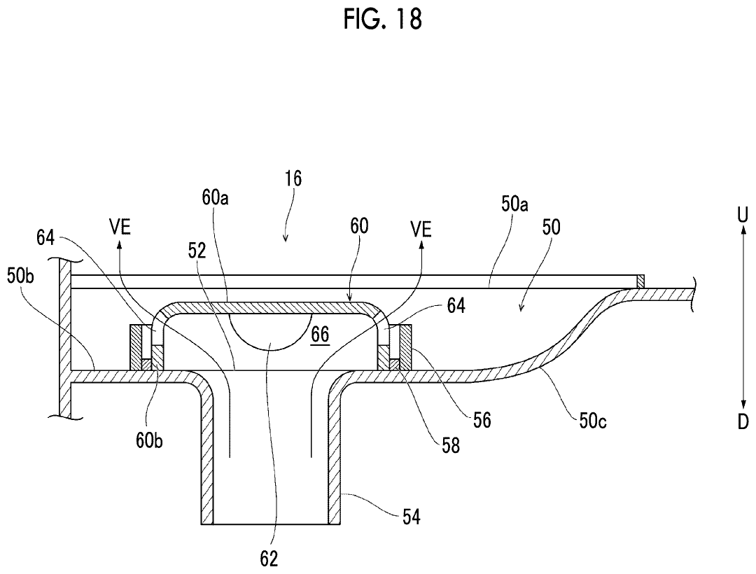

FIG. 18 is a cross-sectional view taken along the line XVIII-XVIII in FIG. 15. As shown in FIG. 18, the inner lid 60 is configured with the top surface 60a and the outer side surface 60b and is attached to the recessed bottom portion 50b to cover at least the bottom opening 52. As a result, a ventilation space 66 is formed inside the inner lid 60. The ventilation space 66 communicates the recessed bottom portion 50b (bottom opening 52) and a filter accommodation portion 72 (see FIG. 20) of the rear cover 70, which will be described later, through each of the ventilation holes 64, and constitutes a part of the ventilation path VE described above.

In addition, in the present embodiment, by forming each of the ventilation holes 64 in the outer side surface 60b of the inner lid 60, the washing solution or the like in the washing tank 13 that is ejected to the U direction side from the bottom opening 52 is prevented from directly hitting the deodorizing filter 90 (see FIG. 20) to be described later. The position of the ventilation hole 64 formed in the inner lid 60 is not particularly limited as long as it is possible to prevent the washing solution or the like in the washing tank 13 from directly hitting the deodorizing filter 90.

### Rear Cover 70

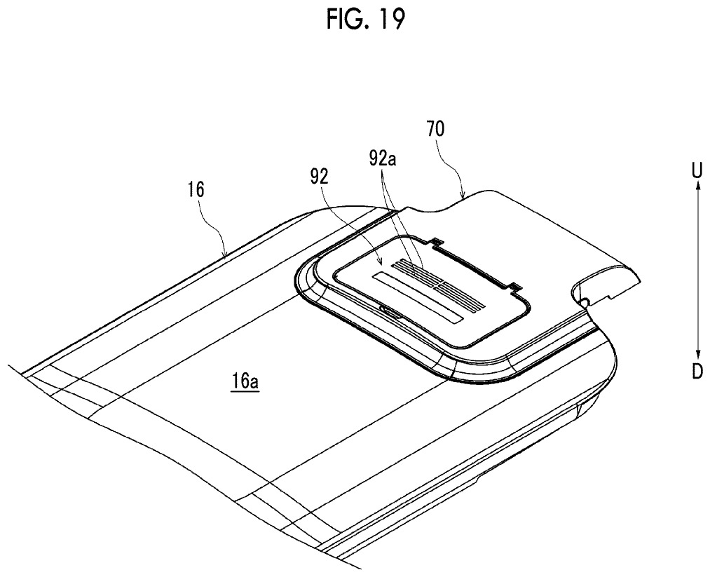

FIG. 19 is an external perspective view of the rear cover 70 and the outer lid 92. FIG. 20 is an exploded perspective view showing a state where the deodorizing filter 90 and the outer lid 92 are detached from the rear cover 70. As shown in FIGS. 19 and 20, the rear cover 70 corresponds to the cover according to the embodiment of the present invention. The rear cover 70 is provided on the front surface portion side of the top cover 16 to cover a part (recessed portion 50) of the top cover 16 and the cover attachment portion 43 and the bracket 43a. The rear cover 70 may cover at least the recessed portion 50. The rear cover 70 is screw-fixed to the top cover 16 from the front surface portion side of the rear cover 70 and the back surface portion side of the top cover 16. The method of fixing the rear cover 70 to the top cover 16 is not particularly limited, and a method other than the screw fixing can also be adopted.

The rear cover 70 is provided with the filter accommodation portion 72 at a position facing the recessed portion 50.

The filter accommodation portion 72 is formed in a substantially recessed shape that is open in the front surface portion side of the rear cover 70 and is recessed toward the D direction side. The above-described inner lid 60 is exposed at the bottom portion of the filter accommodation portion 72, and the deodorizing filter 90 is accommodated in the filter accommodation portion 72 so as to cover the inner lid 60. That is, the filter accommodation portion 72 accommodates the deodorizing filter 90 at a position on the U direction side of the inner lid 60 (a surface side opposite to the surface side facing the recessed bottom portion 50b). The outer lid 92 covers the opening portion of the filter accommodation portion 72.

The outer lid 92 is provided to be rotationally movable with respect to two hinge shafts 73 of the rear cover 70 to be described later, and covers the filter accommodation portion 72, more specifically, the deodorizing filter 90 accommodated in the filter accommodation portion 72. The outer lid 92 is formed with a plurality of ventilation holes 92a constituting a part of the above-described ventilation path VE and two hook portions 93. The area of the outer lid 92 is larger than the area of the deodorizing filter 90 in a case of being viewed from the front surface portion side of the top cover 16.

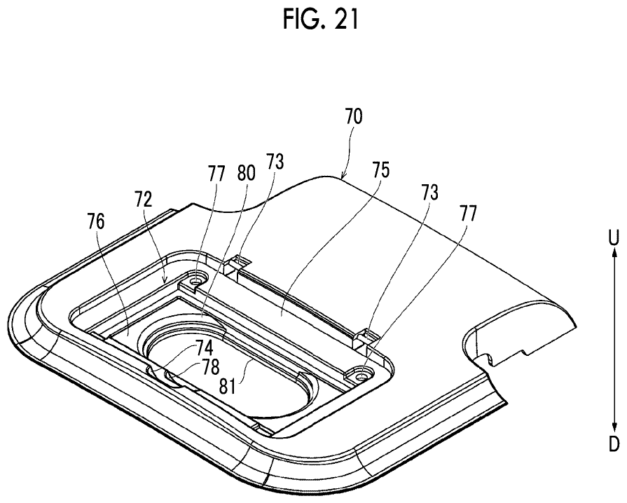

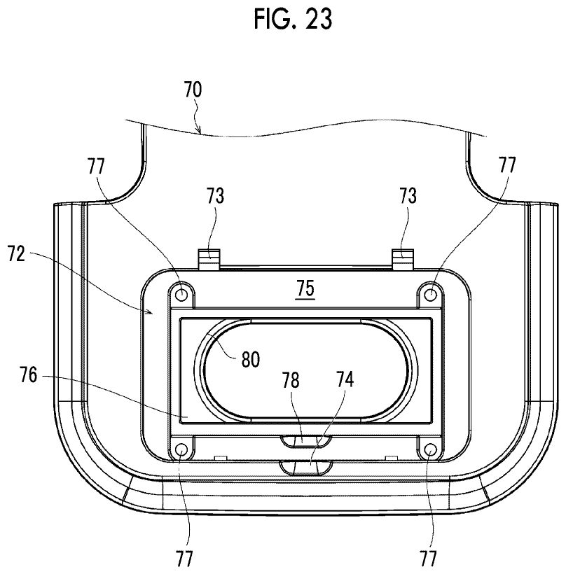

FIG. 21 is an external perspective view of the rear cover 70 as viewed from a front surface portion side. FIG. 22 is an external perspective view of the rear cover 70 as viewed from a back surface portion side. FIG. 23 is a top view of the rear cover 70.

As shown in FIGS. 21 to 23, the rear cover 70 is provided with the two hinge shafts 73 at positions where an opening edge portion of the filter accommodation portion 72 on the rear side is formed. The outer lid 92 is attached to the two hinge shafts 73 to be rotationally movable. In addition, the rear cover 70 is provided with a finger insertion portion 74 at a position where an opening edge portion of the filter accommodation portion 72 on the front side is formed. In a case of opening the outer lid 92, a tip portion of a finger of the operator is inserted into the finger insertion portion 74.

The outer recessed portion 75 is formed in the filter accommodation portion 72. The outer periphery of the outer recessed portion 75 has a shape along the outer periphery of the outer lid 92 in the closed position, which will be described later, in a case of being viewed from the front surface portion side of the top cover 16. Accordingly, the outer lid 92 can be fitted to the outer recessed portion 75. A middle recessed portion 76 that accommodates the deodorizing filter 90 in a replaceable manner and a plurality of screw holes 77 for screw-fixing the rear cover 70 to the top cover 16 are formed at a bottom portion of the outer recessed portion 75.

In addition, a finger insertion portion 78 is formed at a position where an opening edge portion on the front side of the middle recessed portion 76 is formed in the bottom portion of the outer recessed portion 75. In a case of detaching the deodorizing filter 90 from the middle recessed portion 76, a tip portion of a finger of the operator is inserted into the finger insertion portion 78.

The middle recessed portion 76 has a smaller area than the outer recessed portion 75 in a case of being viewed from the front surface portion side of the top cover 16, and has a shape along the outer periphery of the deodorizing filter 90. As a result, the deodorizing filter 90 is fitted to the middle recessed portion 76. As a result, the deodorizing filter 90 is replaceably accommodated in the filter accommodation portion 72. An exposure opening portion 80 through which the inner lid 60 is exposed in the middle recessed portion 76 (inside the filter accommodation portion 72) is formed at a bottom portion of the middle recessed portion 76. The inner lid 60 is mounted on the exposure opening portion 80. Therefore, the exposure opening portion 80 corresponds to an inner lid mounting portion according to the embodiment of the present invention.

The exposure opening portion 80 is formed in a shape (here, an oval shape) corresponding to the shape of the inner lid 60 in a case of being viewed from the front surface portion side of the top cover 16. Accordingly, the inside of the filter accommodation portion 72 and the recessed bottom portion 50b (inside the washing tank 13) communicate with each other through the ventilation hole 64 and the exposure opening portion 80 of the inner lid 60.

A second wall portion 81 (see FIG. 22) that is formed in an oval shape along the opening peripheral edge portion of the exposure opening portion 80 and that protrudes to the D direction side (the recessed portion 50 side) is formed on the back surface portion of the rear cover 70. In a case where the rear cover 70 is attached to the top cover 16, the second wall portion 81 is internally fitted to the first wall portion 56 formed in the recessed bottom portion 50b described above. In other words, the first wall portion 56 is externally fitted to the second wall portion 81. In this case, a distal end portion (an end portion on the D direction side) of the second wall portion 81 abuts the packing 58 described above.

### Deodorizing Filter 90

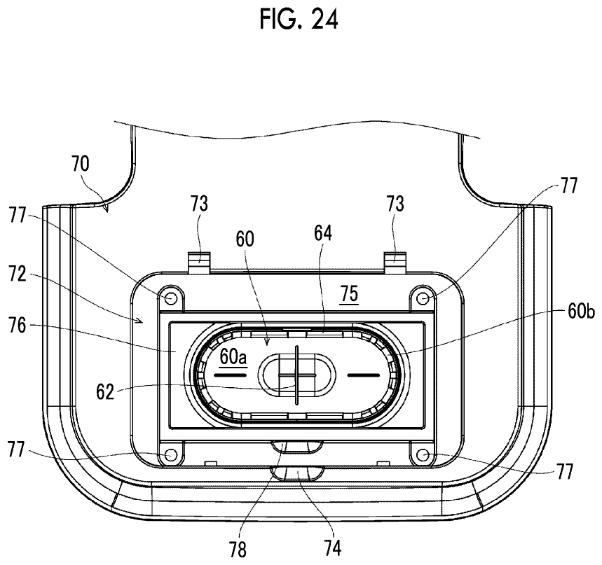

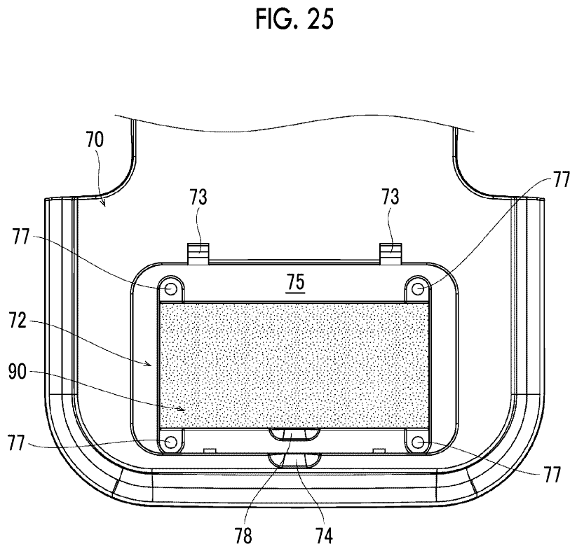

FIG. 24 is a top view of the rear cover 70 before the deodorizing filter 90 is accommodated. FIG. 25 is a top view of the rear cover 70 after the deodorizing filter 90 is accommodated. As shown in FIGS. 24 and 25, the deodorizing filter 90 corresponds to the filter according to the embodiment of the present invention, and is replaceably accommodated in the middle recessed portion 76 so as to cover the inner lid 60.

Since the deodorizing filter 90 is disposed in the middle of the ventilation path VE inside the filter accommodation portion 72, the deodorizing filter 90 deodorizes the odor of the gas discharged from the inside of the washing tank 13 to the outside through the ventilation path VE. Various filters other than the deodorizing filter 90 may be accommodated in the filter accommodation portion 72.

### Outer Lid 92

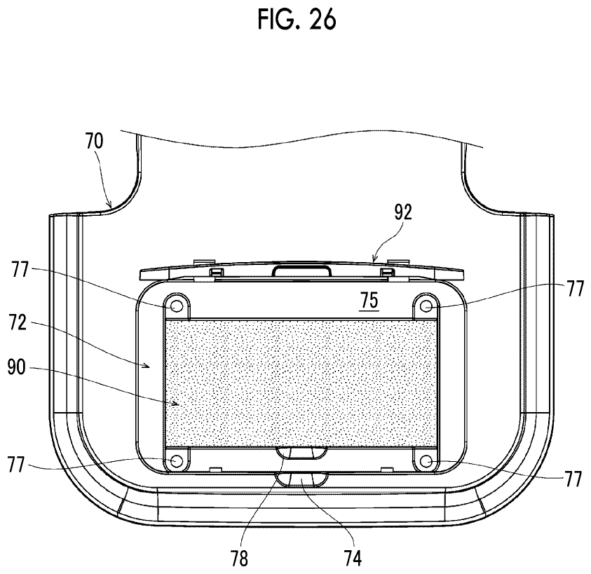

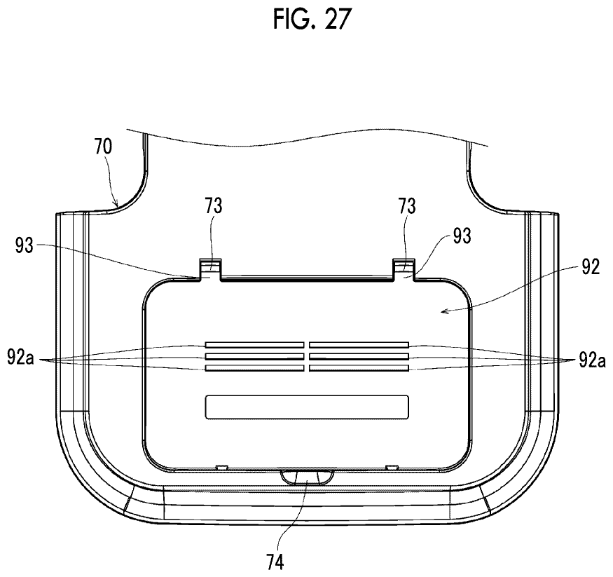

FIG. 26 is a top view of the rear cover 70 in a state where the outer lid 92 is rotationally moved to an open position. FIG. 27 is a top view of the rear cover 70 in a state where the outer lid 92 is rotationally moved to a closed position. As shown in FIGS. 26 and 27, the outer lid 92 is provided with the two hook portions 93 that are engaged with the two hinge shafts 73 to be rotationally movable, respectively. Accordingly, the outer lid 92 is rotationally movable between an open position (see FIG. 26) where the deodorizing filter 90 accommodated in the filter accommodation portion 72 is exposed to the outside and a closed position where the deodorizing filter 90 is fitted to the outer recessed portion 75 and covered. The attachment unit of the outer lid 92 to the rear cover 70 is not limited to the method of using the hinge shaft 73 and the hook portion 93, and various known methods can be adopted.

The deodorizing filter 90 is covered with the outer lid 92 except for a case of replacing the deodorizing filter 90, so that the deodorizing filter 90 is prevented from falling off from the filter accommodation portion 72 in a case where the top cover 16 is rotationally moved to the open position.

The plurality of slit-shaped ventilation holes 92a (corresponding to second ventilation holes according to the embodiment of the present invention) formed in the outer lid 92 constitute a part of the ventilation path VE in a case where the outer lid 92 is in the closed position. The shape and the number of the ventilation holes 92a are not particularly limited.

The gas is discharged to the outside from the inside of the washing tank 13 through the ventilation path VE that extends from the cylindrical portion 54 to the bottom opening 52, the inner lid 60 (ventilation space 66 and ventilation hole 64), the inside of the filter accommodation portion 72 (middle recessed portion 76, deodorizing filter 90, and outer recessed portion 75) and to the ventilation hole 92a.

### Others

The configuration of the apparatus main body 11 (including the washing tank 13) of the washing and disinfection apparatus 10 described in the above-described embodiment is merely an example and can be appropriately changed.

In the above-described embodiment, the washing and disinfection apparatus 10 of the endoscope has been described as an example, but the embodiment of the present invention can be applied to various washing and disinfection apparatuses that wash and disinfect various medical instruments other than the endoscope and lid portions of washing tanks of the washing and disinfection apparatuses.

### APPENDIX

As understood from the description of the embodiment described above in detail, the present specification includes disclosure of various technical ideas including the following invention.

### Supplementary Note 1

A washing and disinfection apparatus for a medical instrument, comprising:
a washing tank that accommodates the medical instrument;
a lid portion that covers an opening portion of the washing tank;
a recessed portion that is provided in a lid main body portion, which faces the washing tank, of the lid portion and includes a bottom portion that is located on a washing tank side with respect to the lid main body portion and a side wall portion that connects the bottom portion and the lid main body portion; and
a bottom opening that is formed in the bottom portion and communicates with the inside of the washing tank, in which
the side wall portion is an inclined portion having a tapered shape from the lid main body portion toward the bottom portion.

### Supplementary Note 2

A lid portion that covers an opening portion of a washing tank in which a medical instrument is accommodated in a washing and disinfection apparatus for a medical instrument, the lid portion comprising:
a lid main body portion;
a recessed portion that is provided in the lid main body portion and includes a bottom portion that is located on a washing tank side with respect to the lid main body portion and a side wall portion that connects the bottom portion and the lid main body portion; and
a bottom opening that is formed in the bottom portion, in which
the side wall portion is an inclined portion having a tapered shape from the lid main body portion toward the bottom portion.

### EXPLANATION OF REFERENCES

10: washing and disinfection apparatus
11: apparatus main body
13: washing tank
13a: washing tank opening portion
13d: peripheral edge portion
13f: bottom front portion
13g: bottom rear portion
13i: slope
13j: terrace portion
16: top cover
16a: cover main body portion
16b: cover outer peripheral portion
16c: rib
17: front panel
18: side panel
19: upper panel
19a: panel opening portion
19b: inclined surface
20: packing
21: net
22: small article washing basket
24: waste liquid outlet
25: circulation port
26: water supply nozzle
27: water level sensor
28a: airtightness test connector
28b: main channel washing connector
28c: sub-channel washing connector
29a: detergent supply nozzle
29b: disinfectant supply nozzle
29c: float switch
33: transparent window
34: paper discharge port
36: operation unit
37: operation button
38: display
39: tag reader
43: cover attachment portion
43a: bracket
43a1: bracket distal end portion
44: foot switch
46: net
50: recessed portion
50a: recessed opening portion
50b: recessed bottom portion
50c: inclined portion
50c1: connection portion
50c2: connection portion
52: bottom opening
54: cylindrical portion
54a: connection portion
56: first wall portion
58: packing
60: inner lid
60a: top surface
60b: outer side surface
62: gripping portion
64: ventilation hole
66: ventilation space
70: rear cover
72: filter accommodation portion
73: hinge shaft
74: finger insertion portion
75: outer recessed portion
76: middle recessed portion
77: screw hole
78: finger insertion portion
80: exposure opening portion
81: second wall portion
90: deodorizing filter
92: outer lid
92a: ventilation hole
93: hook portion
200: top cover
202: protruding portion
204: filter accommodation portion
206: ventilation tubular portion
208: gap
VE: ventilation path

What is claimed is:

1. A washing and disinfection apparatus for a medical instrument, comprising:
a washing tank that accommodates the medical instrument;
a lid portion that covers an opening portion of the washing tank;
a recessed portion that is provided in a lid main body portion of the lid portion and includes a bottom portion and a side wall portion that connects the bottom portion and the lid main body portion; and
a bottom opening that is formed in the bottom portion and communicates with the inside of the washing tank, wherein
the side wall portion is an inclined portion having a tapered shape from the lid main body portion toward the bottom portion.

2. The washing and disinfection apparatus according to claim 1, wherein
a tubular portion extending in a direction from the bottom opening toward the washing tank is provided at the bottom portion.

3. The washing and disinfection apparatus according to claim 1, wherein
a cover that covers the recessed portion is provided on the lid portion.

4. The washing and disinfection apparatus according to claim 3, wherein
the cover is provided with a filter accommodation portion that accommodates a filter.

5. The washing and disinfection apparatus according to claim 4, wherein
an inner lid that covers at least the bottom opening of the bottom portion is provided between the filter accommodation portion and the bottom portion, and
a ventilation space that communicates the filter accommodation portion with the bottom portion is formed in the inner lid.

6. The washing and disinfection apparatus according to claim 5, wherein
the inner lid has a top surface and an outer side surface, and
a first ventilation hole is formed on the outer side surface.

7. The washing and disinfection apparatus according to claim 5, wherein
an inner lid mounting portion on which the inner lid is mounted is provided in the filter accommodation portion.

8. The washing and disinfection apparatus according to claim 5, wherein
the filter accommodation portion accommodates the filter on a surface side opposite to a surface side facing the bottom portion of the inner lid.

9. The washing and disinfection apparatus according to claim 4, wherein
the cover is provided with an outer lid that covers the filter accommodation portion.

10. The washing and disinfection apparatus according to claim 9, wherein
a second ventilation hole is formed in the outer lid.

11. The washing and disinfection apparatus according to claim 1, further comprising:
an apparatus main body that performs washing and disinfection of the medical instrument accommodated in the washing tank, wherein
the lid portion is attached to the apparatus main body to be rotationally movable between an open position where the opening portion is opened and a closed position where the opening portion is closed.

12. A lid portion that covers an opening portion of a washing tank in which a medical instrument is accommodated in a washing and disinfection apparatus for a medical instrument, the lid portion comprising:
a lid main body portion;
a recessed portion that is provided in the lid main body portion and includes a bottom portion and a side wall portion that connects the bottom portion and the lid main body portion; and
a bottom opening that is formed in the bottom portion, wherein
the side wall portion is an inclined portion having a tapered shape from the lid main body portion toward the bottom portion.
